(19) United States
(12) Patent Application Publication
(10) Pub. No.: US 2021/0322126 A1
(43) Pub. Date: Oct. 21, 2021

(54) CONFORMABLE AEROSOL CONTAINMENT DEVICE

(71) Applicant: David Louis Kirschman, Dayton, OH (US)

(72) Inventor: David Louis Kirschman, Dayton, OH (US)

(73) Assignee: Aerobiotix, Inc., Miamisburg, OH (US)

(21) Appl. No.: 17/019,890

(22) Filed: Sep. 14, 2020

Related U.S. Application Data
(60) Provisional application No. 63/010,122, filed on Apr. 15, 2020.

(51) Int. Cl. A61B 90/00; A61B 90/10

(52) U.S. Cl. CPC A61B 90/05; A61B 2560/04; A61B 90/10

(57) ABSTRACT

An aerosol containment enclosure dimensioned to enclose a head of a patient comprising a plurality of panels to be folded or moved from an unfolded position to a folded position in order to form the aerosol containment enclosure to protect from expelled contaminated aerosol particles.

### CROSS-REFERENCE TO RELATED APPLICATION

The present application claims priority to provisional U.S. application Ser. No. 63/010,122 filed Apr. 15, 2020, to which Applicant claims the benefit of the earlier filing date. This provisional application is incorporated herein by reference and made a part hereof.

### BACKGROUND OF THE INVENTION

### 1. Field of the Invention

This application relates to a disposable, substantially cubical enclosure comprising a collapsible framework and a clear polymer window insert.

### 2. Description of the Related Art

Certain medical procedures, particularly endotracheal intubation, cause release of oral and nasal aerosols into the surrounding environment. For patients with communicable disease, this aerosol release can endanger caregivers and surrounding persons. The use of various mechanical barriers to prevent dispersion of bioaerosols have been used, however each has had limitations and impediments to adoption.

What is needed, therefore, is an improved containment system and method that facilitates containing, reducing or eliminating aerosols or preventing them from endangering caregivers.

### SUMMARY OF THE INVENTION

The current invention is a disposable, substantially cubical enclosure comprising a collapsible framework and a clear polymer window insert. The enclosure substantially encloses the patient's head, while comprising ports for the caregiver to manually access the enclosure interior. The outer frame comprises a disposable, flexible material such as corrugated paper or cardboard in the preferred embodiment. The entire device can be shipped and packed in the flat conformation, and quickly folded into its final conformation. The invention further comprises a stabilization means to maintain in its final conformation.

A further embodiment comprises an auxiliary port for the attachment of a powered negative air pressure means.

One object of the invention is to provide an enclosure that is disposable or recyclable and that originates in a generally planar form prior to conformation or folding.

Another object of the invention is to provide an enclosure that facilitates keeping aerosols that are expelled from a patient away from people, such as surgeons, nurses and technicians, so that they are not exposed to such aerosols.

Another object of the invention is to provide an enclosure that is supplied in a generally planar shape, but that is easily conformable or foldable into a final enclosure shape at the time of use.

Still another object of the invention is to provide generally planar pre-folded or conformable enclosure that can be supplied with an inventory of enclosures in a single container, such as a box.

Yet another object of the invention is to provide an enclosure that comprises sealing means or a sealing device for sealing a user's forearms after they have been inserted into the enclosure.

Yet another object of the invention is to provide an enclosure that can be coupled to an auxiliary air pressure means or device and/or a decontamination system, such as by providing a conduit and aperture in the enclosure.

The features of the embodiments described herein may be used alone or in combination with the features of the embodiments shown and described in U.S. Pat. Nos. 9,433,693; 9,457,119; 9,764,054; 10,039,854; 10,532,122 and 10,549,007; as well as U.S. Patent Publication Nos. 2018/0133084; 2018/0133355; 2018/0264391; 2019/0099050 and 2020/0047094, all of which are assigned to the same assignee as the present application and are incorporated herein by reference and made a part hereof, all of which are incorporated herein by reference and made a part hereof. These and other objects and advantages of the invention will be apparent from the following description, the accompanying drawings and the appended claims.

In one aspect, one embodiment of the invention provides an aerosol containment enclosure dimensioned to substantially enclose a head of a patient; the aerosol containment enclosure comprising a frame comprising a plurality of panels, the plurality of panels having a plurality of fold lines or areas of weakness that are adapted to permit the plurality of panels to be folded or moved from an unfolded position to a folded position in order to form the aerosol containment enclosure, at least one of the plurality of panels comprises a polymer window, and at least one of the plurality of panels comprising at least one aperture for permitting access to the patient's head after the aerosol containment enclosure is situated in operative relationship with the patient.

In another aspect, another embodiment of the invention provides a conformable aerosol containment device dimensioned to substantially enclose the head of a patient, comprising a semi-rigid frame comprising linear flexible joints and at least one translucent polymer window; and at least one manual access aperture.

This invention, including all embodiments shown and described herein, could be used alone or together and/or in combination with one or more of the features covered by one or more of the following list of features:
The aerosol containment enclosure wherein the plurality of panels comprise a first plurality of side panels on either side of at least a portion of a main panel, the side panels and a last portion of the main panel being adapted to be folded to form a generally U-shaped first intermediate frame.
The aerosol containment enclosure wherein the plurality of panels comprise a second plurality of side panels, the second plurality of side panels also being adapted to be folded along at least one of the plurality of fold lines or areas of weakness to envelope and enclose the first plurality of side panels to form the aerosol containment enclosure.
The aerosol containment enclosure wherein at least one of the second plurality of side panels comprise at least one locking tab, the main panel comprising at least one locking groove for receiving the at least one locking tab in order to lock the plurality of panels into a final configuration that defines the aerosol containment enclosure.
The aerosol containment enclosure wherein the plurality of panels comprises corrugated paper, cardboard, polymer or other material which will permit relative flexibility without breakage when the plurality of panels are folded along the plurality of lines or areas of weakness while maintaining rigidity in at least one other area of the plurality of panels.
The aerosol containment enclosure wherein the plurality of fold lines or areas of weakness are defined by scored lines or thinned areas in the plurality of panels respectively, or comprises a hinge to permit pivotal movement of at least one of the plurality of panels.
The aerosol containment enclosure wherein the polymer window comprises at least one of a polyethylene sheet or other transparent material that permits visualization of an interior of the aerosol containment enclosure.
The aerosol containment enclosure wherein the polymer window comprises at least one of a polyethylene sheet or other transparent material that permits visualization of an interior of the aerosol containment enclosure, the polymer window being generally planar when in an unfolded form, but generally L- shaped or non-planar after the plurality of panels are in the folded form.
The aerosol containment enclosure wherein at least one aperture is located in the main panel adjacent to the polymer window, the at least one aperture defining at least one access port comprising at least one of a substantially circular, oval, polygonal or rectangular aperture for permitting the placement of a person's hands or forearms therethrough to permit access to an interior area of the aerosol containment enclosure.
The aerosol containment enclosure wherein the access port is fully or partially enclosed by the frame or the polymer window.
The aerosol containment enclosure wherein an integral or non-integral flexible border or sleeve associated with the access port for allowing hands or forearms of various diameters to pass into the interior area of the aerosol containment enclosure while maintaining substantial contact with the forearm to facilitate reducing or preventing aerosols from being exhausted between the forearms and the aerosol containment enclosure.
The aerosol containment enclosure wherein at least one of the plurality of panels comprises at least one port separate from the at least one aperture that receives at least one hand or forearm, the at least one port being adapted and dimensioned to receive at least one of a hose, a duct or airflow device or means for connecting the aerosol containment enclosure to an external air handler or air handling means or system.
The conformable aerosol containment device comprising multiple linear flexible joints which allow for configuration of the device from a substantially flat state to a substantially cubical operational state
The conformable aerosol containment device comprising a stabilizing means, such as at least one slot-in-groove junction to stabilize the device in the substantially cubical operational state.
The conformable aerosol containment device with the semi-rigid frame comprising corrugated paper, cardboard, polymer or other material which will permit relative flexibility without breakage in certain joint areas, and relative rigidity in other areas.
The conformable aerosol containment device with the semi-rigid frame comprising scored, thinned, and/or hinged means to allow for the flexibility.
The conformable aerosol containment device where the at least one translucent polymer window comprises polyethylene sheet or other transparent substance to allow for visualization of the device interior.
The conformable aerosol containment device where the at least one translucent polymer window is flexible and changes conformation based upon the conformation of the semi-rigid frame.
The conformable aerosol containment device where the at least one manual access aperture comprises at least one substantially circular, oval, or rectangular aperture in the frame dimensioned for the placement of the hands and forearms through.
The conformable aerosol containment device wherein the at least one manual access aperture is fully or partially enclosed by the frame and/or the at least one translucent polymer window.
The conformable aerosol containment device wherein the at least one manual access aperture comprises an integral or non-integral flexible border allowing for forearms of various diameters to pass while maintaining substantial contact about the circumference of the forearm.
The conformable aerosol containment device comprising at least one port, physically separate from the aperture for the placement of forearm, dimensioned to receive a hose, duct, or similar airflow means for the purpose of connecting device to external air handling means.

These and other objects and advantages of the invention will be apparent from the following description, the accompanying drawings and the appended claims.

### BRIEF DESCRIPTION OF THE ACCOMPANYING DRAWINGS

FIG. 1 is a perspective view of an enclosure in accordance with one embodiment of the invention;

FIG. 2 is a front view of the enclosure shown in FIG. 1;

FIG. 3 is a plan view of the enclosure of FIG. 1 prior to be conformed or folded;

FIG. 4A is another plan view, similar to FIG. 3, showing further details of the enclosure shown in FIG. 1;

FIG. 4B is a perspective view of the pre-folded or pre-conformed enclosure, showing a plurality of panels MP1, P1 and P2 folded along lines of weakness;

FIG. 4C is a side view of the enclosure shown in FIG. 4B after the panels MP1, P1 and P2 are folded or conformed as shown;

FIG. 5 is another view similar to FIGS. 4C and 4B showing how the panels P1 and P2 are conformed or folded;

FIG. 6A shows another folding or conformable step where panels P3 and P4 are folded over and sandwich panel P1, while panels P5 and P6 are folded over and sandwich panel P2 as shown;

FIG. 6B show the finally conformed and folded enclosure;

FIG. 6C is a sectional view taken along the line 6C-6C in FIG. 6B;

FIG. 7 is an exploded view showing various details of a locking means or device in the form of a tab and groove for securing at least one or a plurality of panels in a locked position;

FIG. 8 is a view illustrating the use of the enclosure with a user and patient, with the enclosure situated about the patient's head;

FIG. 9 is a view showing another embodiment with conformable sleeves or conduits through which a user may palace their forearms into the enclosure, with the sleeves or flexible conduits engaging, gripping and sealing the user's forearms; and

FIG. 10 is a perspective view of another embodiment showing an optional auxiliary port for the attachment of a pressure means or device and/or a decontamination system.

### DETAILED DESCRIPTION OF THE PREFERRED EMBODIMENTS

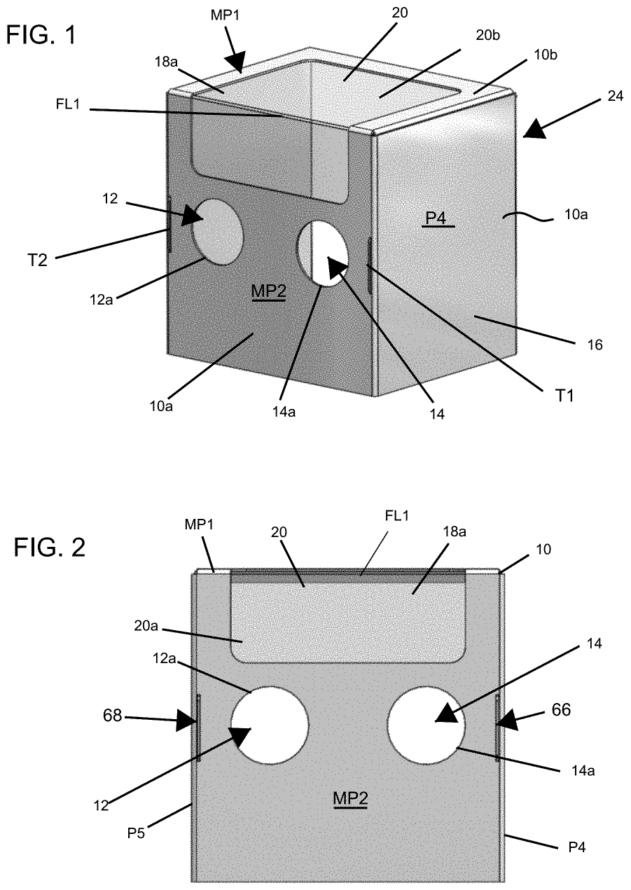

Referring now to FIGS. 1-10, a disposable, recyclable, substantially cubical or polygonal-shaped aerosol containment enclosure 10 is shown. As will be described in more detail later herein relative to FIG. 8, the enclosure 10 encloses a patient's head in order to contain aerosol contaminants that may be expelled by the patient or that may otherwise reside inside the enclosure 10. Note that the enclosure 10 substantially encloses the patient's head during use, yet comprises at least one or a plurality of access ports or apertures 12 and 14 defined by generally circular interior walls 12a and 14a, respectively. It should be understood that the interior walls 12a and 14a may comprise any predetermined shape, such as a circular shape as shown or oval, rectangular or any other polygonal shape. Note that the interior walls 12a and 14a are dimensioned in size to receive hands and forearms of a person, such as a surgeon.

In the illustration being described, the enclosure 10 comprises a generally planar or disposable or recyclable flexible material, such as corrugated paper or cardboard. However, the enclosure 10 could be made of other suitable materials, such as a plastic or polymer that is adapted to be folded and shaped in the manner described herein.

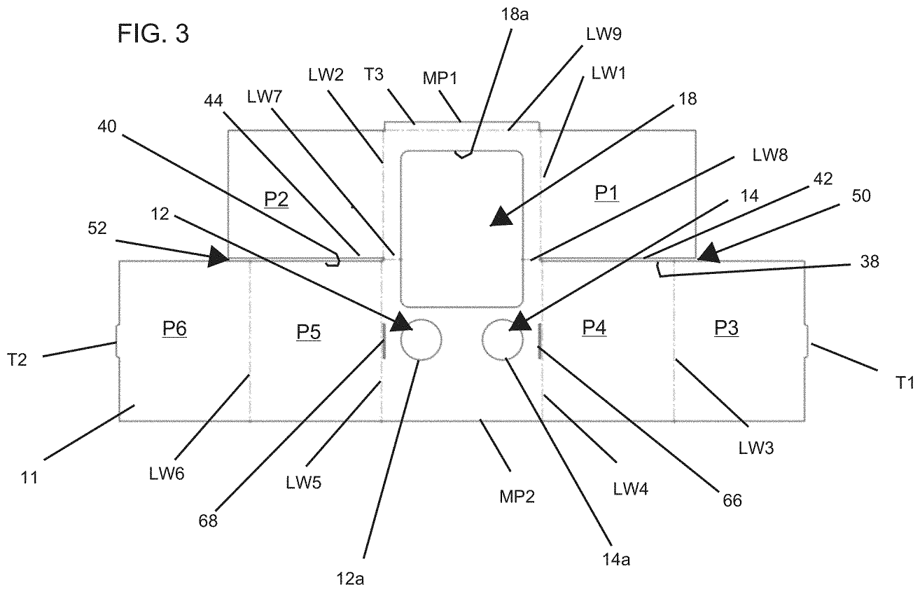

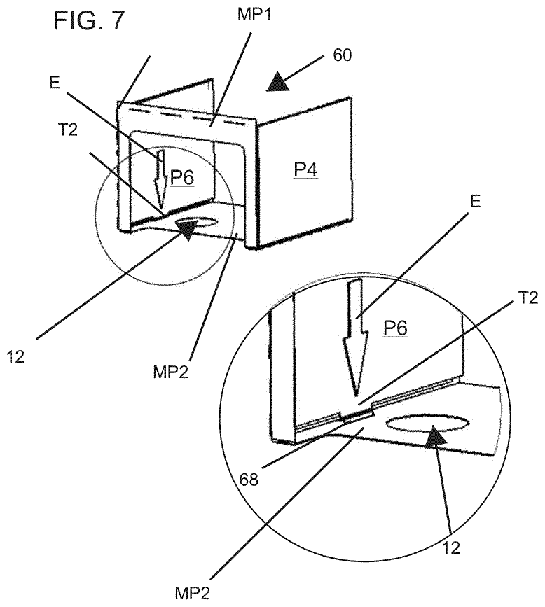

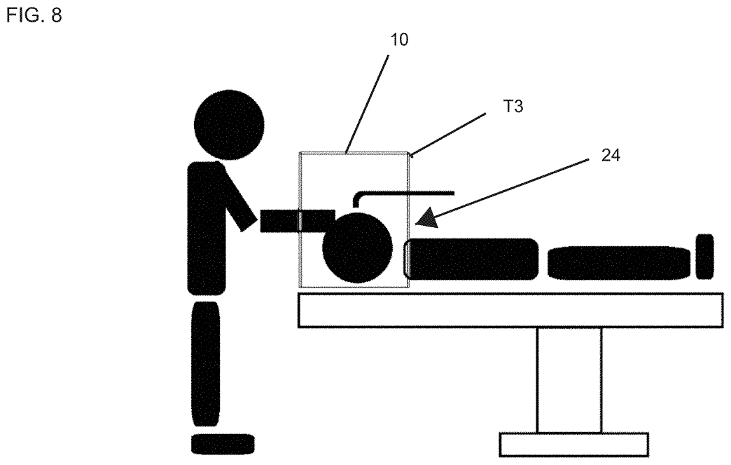

In the illustration being described, the enclosure 10 can be shipped and packed in a flat conformation (FIG. 3) and quickly folded into its final conformation (FIGS. 1 and 7). In this regard, note in FIG. 3 that the enclosure 10 is stamped or cut from a stock (not shown) to provide a material 11 and is in a generally planar form when not in use or prior to use. The planar form facilitates shipment, either alone or with a plurality of other enclosures 10. In the embodiment being described, it is preferred that the enclosure 10 be a disposable and one-time use enclosure, so that after the enclosure 10 is used, for example, in a surgical environment, the enclosure 10 is discarded. Alternatively, it may be recycled for use after decontaminating the enclosure 10.

Note in FIGS. 1 and 2 that the enclosure 10 comprises a plurality of panels P1-P6 that are moveable and a generally centralized main panel MP as shown. The generally centralized main panel MP comprises two areas or sections MP1 and MP2 that are foldable or movable with respect to each other, as best illustrated in FIGS. 3-6C. The enclosure 10 is defined by a frame 16 that is folded to the finished form illustrated in FIGS. 1 and 2. Note that prior to its finished form, the enclosure 10 is provided in a non-folded generally planar form from a supply material, such as the corrugated paper or cardboard mentioned earlier herein. In a preferred embodiment, the enclosure 10 is generally cubical and in its finished and folded form is approximately 20 inches height×20 inches width×15 inches depth. In the illustration being described, the material 11 is corrugated plastic and has a thickness of less than ¼ of an inch and in a preferred embodiment is approximately ⅛ inch thick. Other material options may include corrugated paper, polymer, cardboard, metal or the like.

One procedure for folding from the pre-folded position illustrated in FIG. 3 to the assembled and folded position shown in FIGS. 1 and 2 will now be described. As best illustrated in FIG. 3, note that the material 11 is pre-cut to have the walls 12a and 12b, an area 18 which is defined by an interior wall 18a in the main panel MP. In the illustration being described, the interior wall 18a is generally rectangular and defines the generally rectangular area 18, as illustrated in FIG. 3, but it should be understood that it could take other forms, such as polygonal, curved or circular shapes if desired.

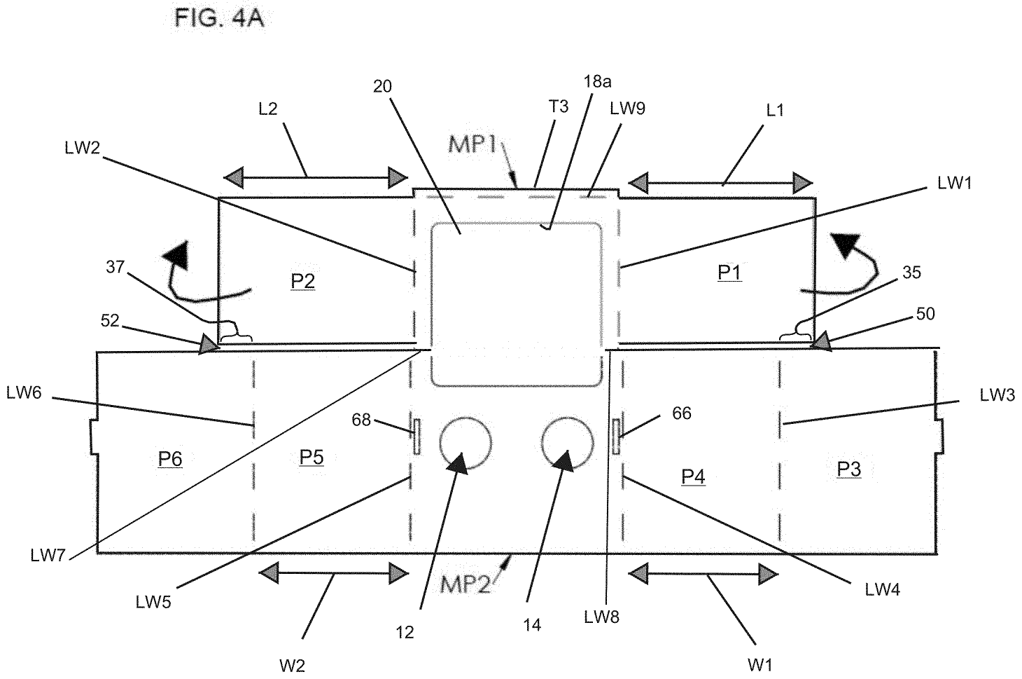

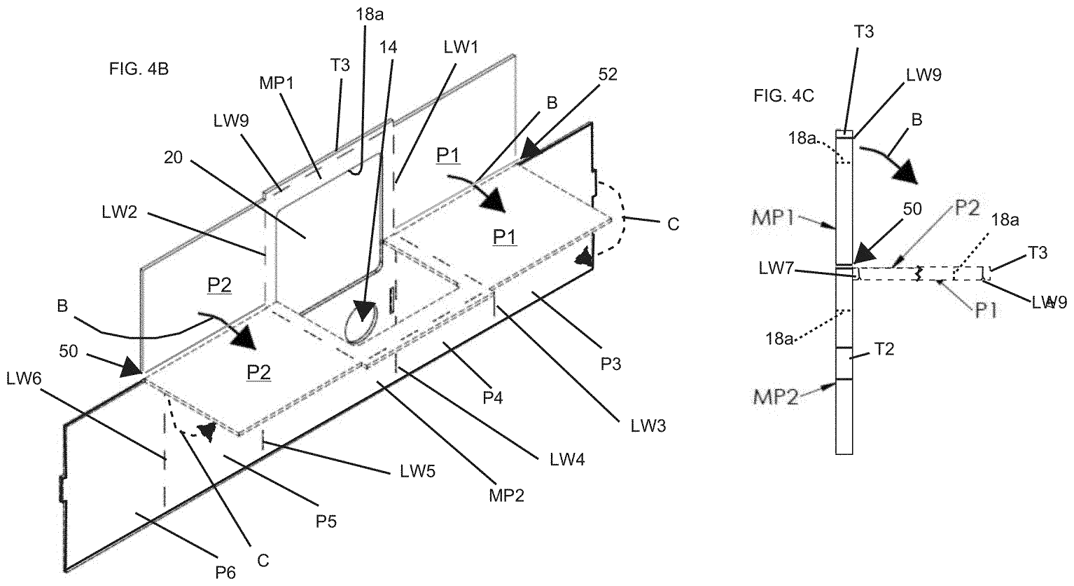

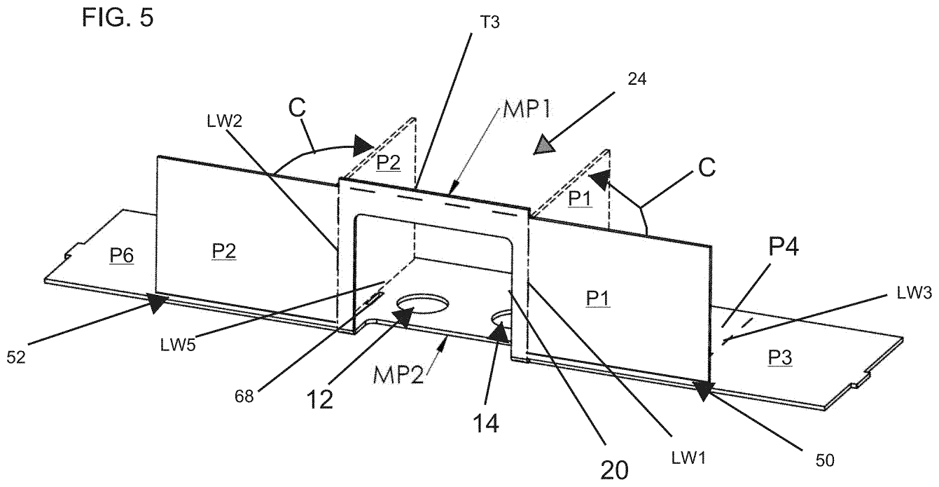

In the illustration being described, the enclosure 10 comprises at least one polymer window 20 situated in or on at least one of the plurality of panels P1-P6 or the main panel MP. In the illustration being described, the interior wall 18a extends primarily from the first main panel MP1 and into the second main panel MP2 as shown. As best illustrated in FIG. 1, after the panels P1, MP1 and P2 are folded in a manner described herein relative to FIGS. 4A-4B, the polymer window 20 also folds along a fold line FL1 (FIG. 1) in order to provide a window that is visible from both a side wall 10a (in the illustration in FIG. 1) of panel MP1 and a plan or top surface 10b of the panel MP2 also as illustrated in FIG. 1. In the illustration being described, the polymer window 20 is secured to the enclosure 10 by conventional means, such as by an adhesive, tape, hot glue or the like. In the embodiment being described, the polymer window 20 comprises a polyethylene sheet or other transparent substance material that allows for visualization into the enclosure 10 (FIGS. 1, 5B and 5C). For ease of illustration and description, the window 20 is shown in FIGS. 1, 2, 9 and 10 and is not shown in FIGS. 3-8 so that the components are visible.

Advantageously, the polymer window 20 is flexible and is adapted to be folded along fold line FL1 (FIGS. 1 and 4A) as illustrated herein so that at least portions of the polymer window 20 lie in a plurality of different planes so that an interior area 24 can be visualized and observed, such as in order to view the patient (FIG. 7) when looking through at least one portion 20a of the polymer window 20 or looking through at least one portion 20b of the polymer window 20 through the panel MP1. Thus, it should be understood that the polymer window 20 is flexible and adjustable and changes conformation based upon the conformation of the semi-rigid frame and in the embodiment being described, the conformation or relative movement of the panels MP1 and MP2 with respect to each other. Although not shown, the enclosure 10 and the material 11 may comprise other polymer windows 20 if desired.

As mentioned earlier herein, the access ports or apertures 12 and 14 are substantially circular, oval or generally rectangular or other polygonal shape and are dimensioned for the placement of hands and forearms therethrough. It should be understood that the apertures 12 and 14 are dimensioned for the placement of the hands and forearms therethrough and note that the apertures 12 and 14 in the illustration being described are completely enclosed by the walls 12a and 14a and/or the polymer window 20, which covers the generally rectangular aperture or area 18 defined by the interior wall 18a. As mentioned earlier, each of the apertures 12, 14 and 18 could have different shapes if desired.

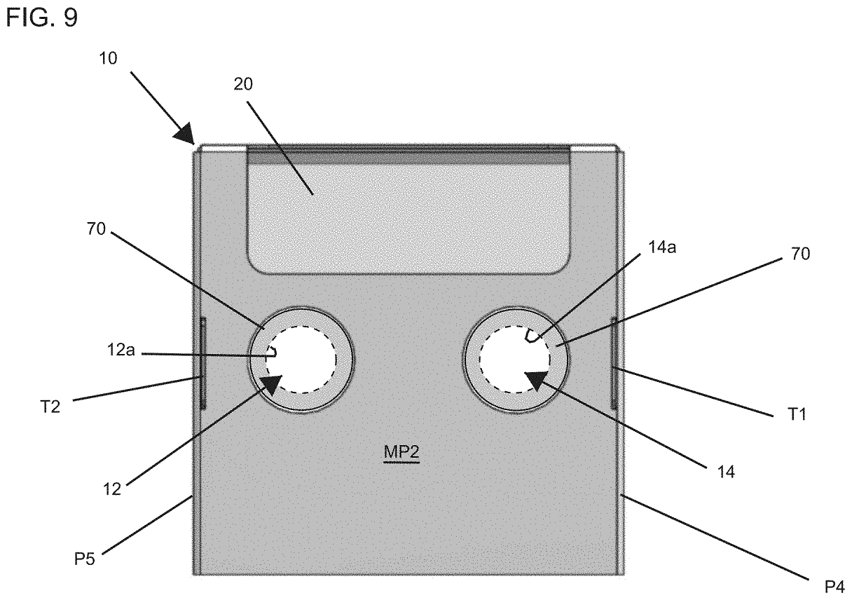

Referring to FIG. 9, notice that the enclosure 10 may comprise an integral or non-integral, flexible border material, sleeves or area adapted to receive and allow forearms and hands of various diameters or sizes to pass into the interior area 24 while maintaining substantial contact about the circumference of the user's forearms to bear against them and provide a seal. In this regard, a flexible material, such as a polymer or paper sleeve having an elastic band that captures and engages a user's forearms so that there is no gap between the walls 12a and 14a and the user's forearms. Thus, the enclosure 10 comprises sealing means or a sealing device for sealing a user's forearms after they have been inserted into the enclosure 10. For example, the walls 12a and 14a may comprise a flexible elastic material 30 that is coupled to the second main panel MP2 and that permit access into and through the apertures 12 and 14, respectively.

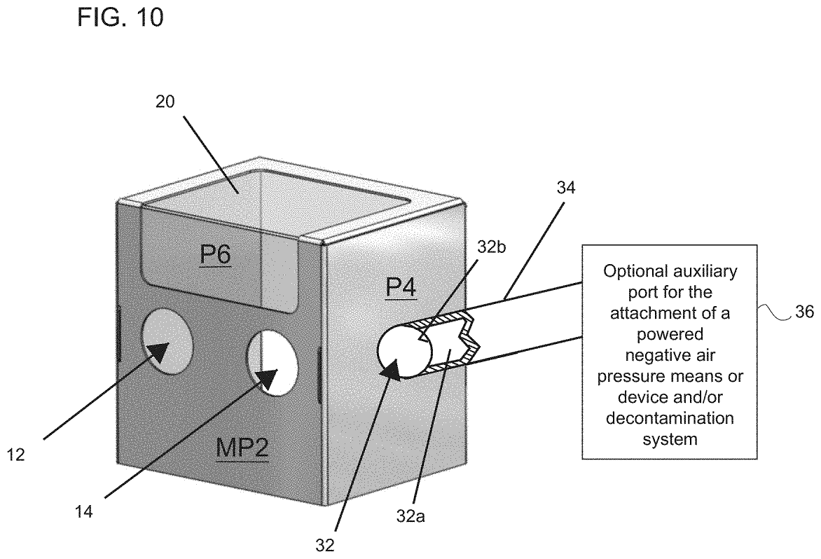

FIG. 10 illustrates another embodiment wherein the enclosure 10 comprises an auxiliary aperture or port 32a defined by a wall 32b that is adapted to be connected to a conduit 34 which in turn is coupled to a powered air pressure device or means 36 for providing either negative air pressure or positive air pressure to the patient. In the preferred embodiment, the powered pressure device or means 36 is a negative air pressure device or means, such as a decontamination or vacuum device for vacuuming the interior area 24 of aerosols and subsequently removing them from the interior area 24 into a device where the aerosol can be decontaminated or treated. In this regard, the ILLUVIA® decontamination air handler product available from Aerobiotix, Inc. of Miamisburg, Ohio may be utilized and could also include the system, apparatus or features of the air handler and irradiation devices shown in U.S. Pat. Nos. 9,433,693; 9,457,119; 9,764,054; 10,039,854; 10,532,122 and 10,549,007; as well as U.S. Patent Publication Nos. 2018/0133084; 2018/0133355; 2018/0264391; 2019/0099050 and 2020/0047094, all of which are assigned to the same assignee as the present application and are incorporated herein by reference and made a part hereof. In one illustrative embodiment, the enclosure 10 is attached to a GERMVAC^{SM} system, which comprises a flexible intake hose for coupling to the enclosure 10. The GERMVAC^{SM} system is also available from Aerobiotix, Inc. located in Miamisburg, Ohio.

It is important to note that the material 11 in the unfolded form (FIG. 3) comprises a plurality of fold lines or lines of weakness that are adapted to permit the plurality of panels P1-P6 and panels MP1 and MP2 to be folded or moved from an unfolded position to a folded position in order to form the enclosure 10. For example, note the lines of weakness LW1 between panel P1 and panel MP1 and line of weakness LW2 between panels P2 and MP1. Likewise, panels P3, P4, MP2, P5 and P6 also have lines of weakness LW3, LW4, LW5, LW5, LW6, LW7 and LW8 as best illustrated in FIG. 3. In the embodiment being described, note that the material 11 generally is provided in a generally flat and generally planar pre-cut or die-cut form as illustrated in FIG. 3 and then subsequently assembled or folded into the final enclosure 10 form, preferably, at the time of use. In the illustration being described, the lines of weakness LW1-LW8 define flexible joints or joint areas that provide and permit relative flexibility between adjacent panels while permitting relative rigidity in other areas so that after the material 11 is folded into its final form, the enclosure 10 defines a generally or substantially rigid cube or enclosure that can be situated over the patient's head. In the illustration being described, the lines of weakness LW1-LW8 are scored or thin areas in a thickness of the material 11 to provide or define hinges or means to allow for the flexibility and movement of the panels P1-P6 and MP1 and MP2.

A procedure for assembling or conforming the material 11 from the generally planar form illustrated in FIG. 3 to the finished form illustrated in FIG. 1 will now be described relative to FIGS. 4A-6C.

Referring now to FIGS. 3-6C, one illustrative procedure for the material 11 into the finished substantially cubical form in FIG. 1 will now be described. Of course, other procedures can be used as will become apparent to the skilled artisan. It should be understood that each panel P1 and P2 has an edge 38 and 40 which is situated generally adjacent and opposite the edge 42 and 44, respectively. Note that the panels P1 and P2 extend slightly beyond a width W1 (FIG. 4A) and W2, respectively, of panels P4 and P5. A small portion of the panels P1 and P2 (identified with arrows 35 and 37, respectively) also extend across and is situated opposed to at least a portion of panels P3 and P6 as shown. The panels P1 and P2 have a length L1 and length L2, respectively, that is substantially the same or similar so that when the panels P1 and P2 are folded, they will form and become the side walls 10a and 10b, respectively, of the enclosure 10 as described herein.

It is important to note that there is a first gap 50 between the panels P1 and panels P3 and P4 and a second gap 52 between the panel P2 and panels P5 and P6, as best illustrated in FIG. 3, which makes them easily conformable. In a first preferred step, the panel MP1 and MP2 are folded downward as shown in FIGS. 4A-4C along the lines of weakness LW7 and LW8 from the position shown in FIG. 4A in the direction of arrow B in FIG. 4B until the panels are situated generally perpendicular or orthogonal to the lower panels P3-P6 as illustrated in FIGS. 4B and 4C.

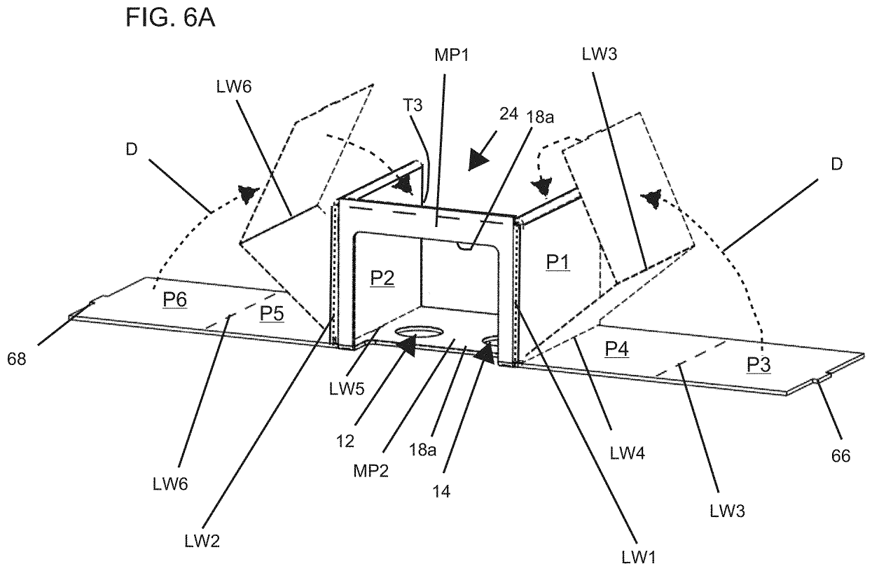

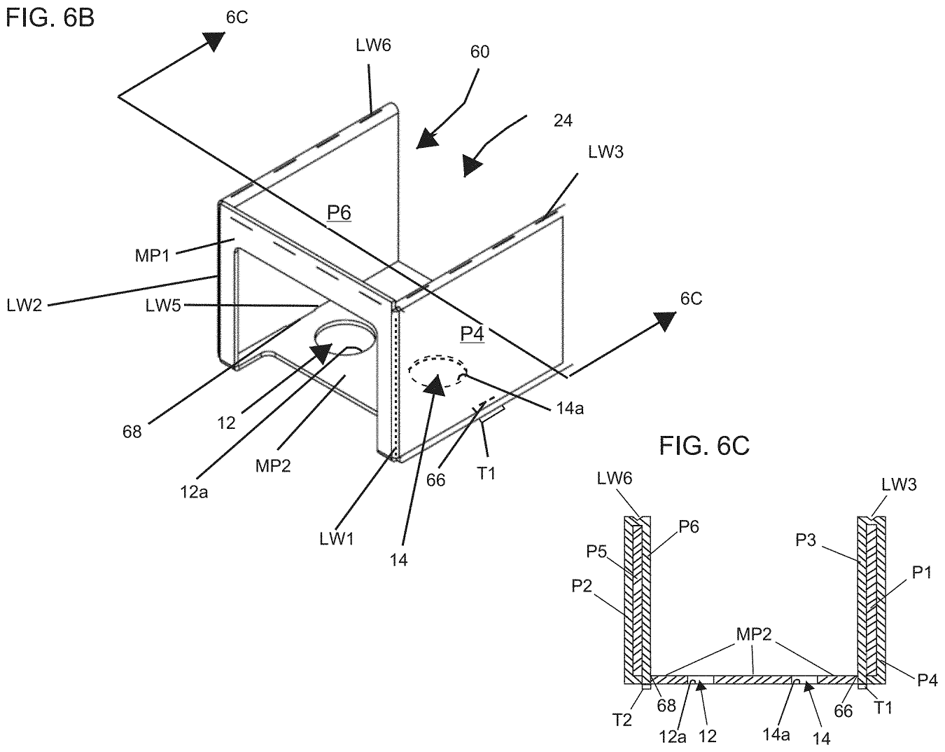

Once the panels MP1, P1 and P2 are folded or conformed as described, the conforming and assembly routine proceeds as shown in FIG. 5 whereupon the panels P1 and P2 are folded along the lines of weakness LW1 and LW2 and in the direction of arrow C in FIG. 5. Note the panels are shown in the folded position in phantom in FIG. 5 and note that the panels P1, P2 and MP1 form a general U-shape as best illustrated in FIGS. 5 and 6A and cooperate to define the interior area 24.

Referring to FIGS. 6A-6C, the routine proceeds whereupon the panels P3 and P4 and panels P5 and P6 are moved in the direction of arrows D (FIG. 6A) and are received in the interior area 24 as illustrated in FIGS. 6A-6C. After the panels P3-P6 are conformed or folded and panels P3 and P6 are received in the interior area 24, notice how the panels P3 and P4 capture or sandwich and receive the side panel P1. Likewise, the panels P5 and P6, after being folded such that panel P6 is in the interior area 24, capture and sandwich the panel P2 as shown in FIGS. 6B and 6C.

The system and enclosure 10 comprises stabilizing and locking means or a locking device. In one embodiment, the enclosure 10 includes at least one or a plurality of tabs T1 and T2 that are received in generally complementary slots and grooves 66, 68 (FIGS. 2 and 6C) that facilitate stabilizing the enclosure 10 in its substantially cubical conformation. Thus, note in the FIGS. 3, 5 and 6A-7 that grooves 66 and 68 capture the tabs T1 and T2, respectively, after the panels P3, P4, P5 and P6 are folded as mentioned earlier. Likewise, the tab T2 on panel P6 is received in the mating slot or groove 68 (FIG. 3) thereby locking the panels P5 and P6 onto the panel P2 as shown.

Advantageously, the preformed material 11 is die cut or pre-cut with the tabs T1 and T2 and their mating slots or grooves 66, 68, respectively, so that no further means or apparatus are necessary to maintain the enclosure 10 in the locked and assembled frame as shown. FIG. 7 further illustrates this point further. Note that the panel P7 is moved downward in the direction of arrow E in FIG. 7 until the tab T2 is received in the mating slot or groove 68.

As best illustrated in FIG. 3, the main panel MP1 also comprises an elongated tab T3 (FIG. 3). During use, the enclosure 10 is situated over the patient's head as illustrated in FIG. 8. The tab T3 can be folded along the line of weakness LW9 (FIG. 3) such that it is angled downward (as viewed in FIG. 8). The tab T3 defines or provides the deflector or deflection means for deflecting the aerosols exhausted from a patient downward before they exit the interior area 24 of the enclosure 10. This further facilitates preventing unwanted aerosols from being directed upwards and toward personnel, such as surgeons, nurses and the like.

FIG. 8 illustrates the use of the enclosure 10 with a user and patient, with the enclosure 10 situated about the patient's head. Now that the enclosure 10 is fully assembled it can be placed in operative relationship with a patient by situating the enclosure 10 so that the patient's head is received or situated in the interior area 24. After the enclosure 10 is placed in proximity or even over the patient's head, the polymer window 20 can be used to view the patient's head while a procedure is performed and while, for example, a person (such as a surgeon) has their hands and forearms inside the apertures 12 and 14.

As mentioned earlier herein, the system and enclosure 10 may further comprise an integral or non-integral flexible and/or elastic sealing collar 70 situated or secured in proximity to the apertures 12 and 14 as illustrated in FIG. 9. The user, such as a surgeon, places his or her hands inside the flexible and/or elastic collar 70 and the elastic or other means secures and seals the flexible and/or elastic collar 70 to the user's forearm so that aerosols cannot escape from between the arms of the user and the walls 12a and 14a of the main panel MP1. This facilitates unwanted aerosols or contaminants from escaping the enclosure 10 and reaching the user.

To further facilitate removal of aerosols and contaminants, the system and enclosure 10 further comprises the optional auxiliary port 32a (FIG. 10) mentioned earlier herein. The optional auxiliary port 32a provides an aperture 32 to which a conduit 34, such as a duct, tube, passageway, hose or similar conduit that is adapted to connect to the external air pressure device or means 36. As mentioned earlier, the powered pressure device or means 36 may be a positive pressure device but, in a preferred embodiment, it is a negative air pressure or vacuum that vacuums the air out of the interior area 24. In one embodiment, the powered pressure device or means 36 may have means and an apparatus for decontaminating the aerosol received from the enclosure 10. As mentioned earlier, the ILLUVIA system or other system is available from Aerobiotix, Inc. of Miamisburg, Ohio , as mentioned earlier herein.

### Other Considerations and Advantages

1. Advantageously, the system, method and apparatus and means described herein are adapted to provide an inexpensive, disposable, easy to assemble and substantially cubical enclosure 10 comprising a collapsible framework and clear polymer window 20 insert. During use, the patient is situated on the room table in one illustrative embodiment (FIG. 7) and the enclosure 10 is situated about or around the patient's head. The surgeon uses his or her hands, inserted through the apertures 12 and 14, to perform a procedure on the patient. Notice that the polymer window 20 is generally L-shaped when fully assembled which provides the user with the ability to look straight down to view the patient or to view the patient from the side of the main panel MP2.

2. After the enclosure 10 is used, it can simply be discarded, or alternatively, decontaminated and then recycled.

3. The entire enclosure 10 can be shipped and packed in the flat conformation shown in FIG. 3. It is also contemplated that a plurality of the flat enclosures 10 may be packaged in a single container, such as a box, and shipped to an end user who can use the box for storing the inventory of enclosures 10 until each one is needed and consumed. It is contemplated that the enclosure 10 would be a single use and throw-away enclosure 10 in the preferred embodiment. However, it is also contemplated that a user may obtain multiple uses of the enclosure 10 if it is adequately sanitized between uses.

4. Advantageously, the system and enclosure 10 provide means for reducing or eliminating aerosol releases that can endanger caregivers and surrounding persons, such as surgeons, nurses, technicians and the like.

5. In conjunction with a negative air unit, the system and enclosure 10 can create a localized negative pressure environment in areas where negative pressure rooms are not available.

6. Due to the disposable nature of the system and enclosure 10, the cleaning of highly decontaminated fixed, prior-art shields is eliminated.

7. The system and enclosure 10 can also comprise ports or access panels for the placement of instrumentation, such as breathing tubes, cameras, transducers, etc.

This invention, including all embodiments shown and described herein, could be used alone or together and/or in combination with one or more of the features covered by one or more of the claims set forth herein, including but not limited to one or more of the features or steps mentioned in the Summary of the Invention and the claims.

While the system, apparatus and method herein described constitute preferred embodiments of this invention, it is to be understood that the invention is not limited to this precise system, apparatus and method, and that changes may be made therein without departing from the scope of the invention which is defined in the appended claims.

What is claimed is:

1. An aerosol containment enclosure dimensioned to substantially enclose a head of a patient; said aerosol containment enclosure comprising:
a frame comprising a plurality of panels, said plurality of panels having a plurality of fold lines or areas of weakness that are adapted to permit said plurality of panels to be folded or moved from an unfolded position to a folded position in order to form the aerosol containment enclosure;
at least one of said plurality of panels comprises a polymer window; and
at least one of said plurality of panels comprising at least one aperture for permitting access to the patient's head after the aerosol containment enclosure is situated in operative relationship with said patient.

2. The aerosol containment enclosure as recited in claim 1, wherein said plurality of panels comprise a first plurality of side panels on either side of at least a portion of a main panel, said side panels and a last portion of said main panel being adapted to be folded to form a generally U-shaped first intermediate frame.

3. The aerosol containment enclosure as recited in claim 2, wherein said plurality of panels comprise a second plurality of side panels, said second plurality of side panels also being adapted to be folded along at least one of said plurality of fold lines or areas of weakness to envelope and enclose said first plurality of side panels to form said aerosol containment enclosure.

4. The aerosol containment enclosure as recited in claim 3, wherein at least one of said second plurality of side panels comprise at least one locking tab, said main panel comprising at least one locking groove for receiving said at least one locking tab in order to lock said plurality of panels into a final configuration that defines said aerosol containment enclosure.

5. The aerosol containment enclosure as recited in claim 1, wherein said plurality of panels comprises corrugated paper, cardboard, polymer or other material which will permit relative flexibility without breakage when said plurality of panels are folded along said plurality of lines or areas of weakness while maintaining rigidity in at least one other area of said plurality of panels.

6. The aerosol containment enclosure as recited in claim 1, wherein said plurality of fold lines or areas of weakness are defined by scored lines or thinned areas in said plurality of panels respectively, or comprises a hinge to permit pivotal movement of at least one of said plurality of panels.

7. The aerosol containment enclosure as recited in claim 1, wherein said polymer window comprises at least one of a polyethylene sheet or other transparent material that permits visualization of an interior of the aerosol containment enclosure.

8. The aerosol containment enclosure as recited in claim 1, wherein said polymer window comprises at least one of a polyethylene sheet or other transparent material that permits visualization of an interior of the aerosol containment enclosure, said polymer window being generally planar when in an unfolded form, but generally L-shaped or non-planar after said plurality of panels are in said folded form.

9. The aerosol containment enclosure as recited in claim 2, wherein at least one aperture is located in said main panel adjacent to said polymer window, said at least one aperture defining at least one access port comprising at least one of a substantially circular, oval, polygonal or rectangular aperture for permitting the placement of a person's hands or forearms therethrough to permit access to an interior area of said aerosol containment enclosure.

10. The aerosol containment enclosure as recited in claim 9, wherein said access port is fully or partially enclosed by said frame or said polymer window.

11. The aerosol containment enclosure as recited in claim 9, wherein an integral or non-integral flexible border or sleeve associated with said access port for allowing hands or forearms of various diameters to pass into said interior area of said aerosol containment enclosure while maintaining substantial contact with said forearm to facilitate reducing or preventing aerosols from being exhausted between the forearms and said aerosol containment enclosure.

12. The aerosol containment enclosure as recited in claim 1, wherein at least one of said plurality of panels comprises at least one port separate from said at least one aperture that receives at least one hand or forearm, said at least one port being adapted and dimensioned to receive at least one of a hose, a duct or airflow device or means for connecting said aerosol containment enclosure to an external air handler or air handling means or system.

13. A conformable aerosol containment device dimensioned to substantially enclose the head of a patient, comprising:
a semi-rigid frame comprising linear flexible joints;
at least one translucent polymer window; and at least one manual access aperture.

14. The conformable aerosol containment device of claim 13 comprising multiple linear flexible joints which allow for configuration of the device from a substantially flat state to a substantially cubical operational state

15. The conformable aerosol containment device of claim 14 comprising a stabilizing means, such as at least one slot-in-groove junction to stabilize the device in said substantially cubical operational state.

16. The conformable aerosol containment device of claim 13 with said semi-rigid frame comprising corrugated paper, cardboard, polymer or other material which will permit relative flexibility without breakage in certain joint areas, and relative rigidity in other areas.

17. The conformable aerosol containment device of claim 16 with said semi-rigid frame comprising scored, thinned, and/or hinged means to allow for said flexibility.

18. The conformable aerosol containment device of claim 13 where said at least one translucent polymer window comprises polyethylene sheet or other transparent substance to allow for visualization of the device interior.

19. The conformable aerosol containment device of claim 18 where the said at least one translucent polymer window is flexible and changes conformation based upon the conformation of said semi-rigid frame.

20. The conformable aerosol containment device of claim 13 where said at least one manual access aperture comprises at least one substantially circular, oval, or rectangular aperture in said frame dimensioned for the placement of the hands and forearms through.

21. The conformable aerosol containment device of claim 20 wherein said at least one manual access aperture is fully or partially enclosed by said frame and/or said at least one translucent polymer window.

22. The conformable aerosol containment device of claim 20 wherein said at least one manual access aperture comprises an integral or non-integral flexible border allowing for forearms of various diameters to pass while maintaining substantial contact about the circumference of said forearm.

23. The conformable aerosol containment device of claim 13 comprising at least one port, physically separate from said aperture for the placement of forearm, dimensioned to receive a hose, duct, or similar airflow means for the purpose of connecting device to external air handling means.
